(19) United States
(12) Patent Application Publication
(10) Pub. No.: US 2007/0048823 A1
(43) Pub. Date: Mar. 1, 2007

(54) Compositions and Methods for Producing Apolipoprotein Gene Products in Lactic Acid Bacteria

(75) Inventor: Jean-Louis Dasseux, Vieille-Toulouse (FR)

Correspondence Address: DECHERT LLP, P.O. BOX 10004, PALO ALTO, CA, 94303, US

(73) Assignee: Cerenis Therapeutics, S.A., Labege cedex (FR)

(21) Appl. No.: 11/467,500

(22) Filed: Aug. 25, 2006

Related U.S. Application Data
(60) Provisional application No. 60/712,295, filed on Aug. 26, 2005.

(51) Int. Cl. C07K 14/775; C07H 21/04; C12P 21/06; C12N 1/21; C12N 15/74

(52) U.S. Cl. 435/69.1; 435/471; 435/252.3; 530/359; 536/23.5

(57) ABSTRACT

The present disclosure relates to compositions and methods for producing recombinant apolipoproteins in lactic acid bacteria.

### 1. CROSS REFERENCE TO RELATED APPLICATIONS

This application claims benefit under 35 U.S.C. § 119(e) to U.S. application Ser. No. 60/712,295, filed Aug. 26, 2005, the contents of which are incorporated herein by reference.

### 2. BACKGROUND

Circulating cholesterol is carried by two major cholesterol carriers, low density lipoproteins (LDL) and high density lipoproteins (HDL). LDL is believed to be responsible for the delivery of cholesterol from the liver (where it is synthesized or obtained from dietary sources) to extrahepatic tissues in the body. It is believed that plasma HDL particles play a major role in cholesterol regulation, acting as scavengers of tissue cholesterol.

Atherosclerosis is a progressive disease characterized by the accumulation of cholesterol within the arterial wall. The lipids deposited in atherosclerotic lesions are derived primarily from plasma LDL; thus, LDLs have popularly become known as the “bad” cholesterol. In contrast, HDL serum levels correlate inversely with coronary heart disease, and as a consequence; high serum levels of HDL are regarded as a negative risk factor. Thus, HDL has popularly become known as the “good” cholesterol.

Recent studies of the protective mechanism(s) of HDL have focused on apolipoprotein A-I (ApoA-I), the major component of HDL. High plasma levels of ApoA-I are associated with absence or reduction of coronary lesions (Maciejko et al., 1983, N Engl J Med 309:385-89; Sedlis et al., 1986, Circulation 73:978-84). However, the therapeutic use of ApoA-I and known variants of ApoA-I, as well as reconstituted HDL, is limited by the large amount of apolipoprotein required for therapeutic administration and by the cost of protein production, considering the low overall yield of production. Thus, there is a need to develop alternative methods for the production of ApoA-I that can be used to treat and/or prevent cholesterol accumulation within coronary arteries.

### 3. SUMMARY

The present disclosure provides compositions and methods for producing recombinant apolipoproteins free from endotoxin. Recombinant apolipoproteins that can be made using the methods described herein include, but are not limited to, preproapolipoprotein forms of ApoA-I, ApoA-II, ApoA-IV, ApoA-V and ApoE; pro- and mature forms of human ApoA-I, ApoA-II, ApoA-IV, and ApoE; and active polymorphic forms, isoforms, variants and mutants as well as truncated forms, the most common of which are ApoA-I_{M} (ApoA-I_{M}) and ApoA-I_{P} (ApoA-I_{P}).

In some aspects, the present disclosure provides expression vectors, host cells comprising the expression vectors, and methods of using the vectors for producing an apolipoprotein of interest in a non-endotoxin producing bacteria, such as lactic acid bacteria. Suitable lactic acid bacteria include, but are not limited to, Lactococcus spp., Streptococcus spp., Lactobacillus spp., Leuconostoc spp., Pediococcus spp., Brevibacterium spp. and Propionibacterium spp. Appropriate regulatory nucleotide sequences are operably linked to the nucleotide coding sequence encoding the apolipoproteins to express apolipoprotein in lactic acid bacteria. Regulatory nucleotides sequences include, but are not limited to, constitutive promoters and regulatable, i.e., inducible, promoters. In some embodiments, the regulatory sequences comprise lactic acid bacteria regulatory sequences.

In some aspects, the methods comprise the steps of constructing a recombinant lactic acid bacterium comprising a nucleotide sequence coding for an apolipoprotein, and operably linked thereto, appropriate regulatory nucleotide sequences to control the expression of the coding sequence, cultivating the recombinant lactic acid bacterium under conditions effective to express the apolipoprotein, and recovering the apoliprotein from the lactic acid bacterium or from the culture medium.

The recombinant apolipoproteins can be used to treat and/or prevent a variety of disorders and conditions, including dyslipidemia, and/or the various diseases, disorders and/or conditions associated therewith.

### 4. DETAILED DESCRIPTION

In high-throughput early discovery and high-yield production of candidate therapeutic proteins, E. coli based expression systems are widely used. However, not all proteins can be produced in high yields using E. coli as a host organism. In addition, successful recombinant protein expression/purification in E. coli depends on a high-fidelity system capable of rendering purified proteins free of contaminants, such as endotoxin. The presence of endotoxin in purified protein samples obtained from E. coli is often undetected. Moreover, methods commonly used to remove contaminants, such as anion exchange chromatography, do not remove endotoxins. See, e.g., McKinstry, et al., 2003, Biotechniques 35:724-6.

Production of apolipoprotein A-I in E. coli is low. See, e.g., U.S. Pat. No. 5,059,528 and references cited therein; see also McGuire, et al., 1996, J Lipid Res. 37:1519-1528; Panagotopulos, et al., 2002, Protein Expr Purif. 25:353-61; and Ryan, et al., 2003, Protein Expr Purif. 27:98-103. Purification steps required to remove endotoxin can reduce the yield even more. Depending on the recombinant protein being expressed in E. coli, it may not be possible to eliminate the contaminating endotoxin and achieve a level of purity that complies with current Good Manufacturing Practice (cGMP) . See, e.g., Ma, et al., 2004, Acta Biochim Biophys Sin. 36:419-24. For example, apolipoprotein A-1 binds endotoxin (lipopolysaccharide (LPS)) and neutralizes its toxicity (see, e.g., Ma, et al., 2004, Acta Biochim Biophys Sin. 36(6):419-24). Thus, it may not be possible to develop an E. coli high-fidelity system capable of rendering purified recombinant apolipoproteins free of endotoxin. Production of apolipoproteins in other expression systems, such as yeast and insect cells, is also low. See, e.g., U.S. Pat. No. 5,059,528 and references cited therein.

The present disclosure provides compositions and methods for producing recombinant apolipoproteins in non-endotoxin producing bacteria, such as lactic acid bacteria. Advantages to using non-endotoxin bacteria, such as lactic acid bacteria for the production of apolipoprotein include: (1) the absence of endotoxins, which are components of the cell wall in most gram-negative bacteria, but are not present as a component of the cell wall in lactic acid bacteria; (2) the availability of lactic acid bacterial strains, including L. lactis strains, that do not produce extracellular proteases; (3) ease of manipulating lactic acid bacteria; (4) the ability of lactic acid bacteria to secrete recombinant peptides, polypeptides or proteins, which can be stable and easier to purify; (5) use of fermentative metabolism (i.e., fermentation occurring in the absence of oxygen) that simplifies the scaling up of protein production by reducing or eliminating the need for specially designed equipment needed for avoiding localized pockets of oxygen, which if present, can decrease cell growth and reduce yield; (6) the availability of inducible expression systems for increasing the yields of expressed gene products; and (7) long history of safe use of lactic acid bacteria in the food industry, making them attractive cloning hosts for the production of therapeutic proteins, such as apolipoproteins.

### 4.1 Lactic Acid Bacteria

In view of the above, the present disclosure provides compositions and methods for expressing apolipoprotein in lactic acid bacteria. As used herein the term “lactic acid bacterium” refers to a gram-positive, microaerophilic or anaerobic bacterium that ferments sugars with the production of acids, including lactic acid as the predominantly produced acid. Typically, the methods and compositions employ lactic acid bacteria that are used industrially, such as Lactococcus spp., Streptococcus spp., Lactobacillus spp., Leuconostoc spp., Pediococcus spp., Brevibacterium spp. and Propionibacterium spp. Lactic acid producing bacteria belonging to the strictly anaerobic group, bifidobacteria, i.e., Bifidobacterium spp., which are frequently used as food starter cultures alone or in combination with lactic acid bacteria, can also be included within the lactic acid bacteria family.

It is to be understood that other non-endotoxin producing bacteria, including other gram-positive bacteria known to those of skill in the art, can be used to produce recombinant apolipoproteins, such that the scope of production of recombinant apolipoproteins is not limited to the lactic acid bacteria described above.

Recombinant lactic acid bacteria can be constructed to comprise a nucleotide sequence that codes for an apolipoprotein. The nucleotide sequence that codes for the apolipoprotein can be optionally linked to an appropriate regulatory nucleotide sequence(s) to control the expression of the coding sequence using methods that are well-known in the art (see, e.g., Sambrook et al., 1989, Molecular Cloning: A Laboratory Manual, Cold Spring Harbor, Cold Spring Harbor Laboratory Press, N.Y.). In addition, the codon usage patterns of a number of sequenced genes from different Lactobacillius species have been analyzed, making it possible to develop approaches to bypass codon bias if necessary. See, e.g., Pouwels and Leunissen, 1994, Nucleic Acids Res. 22:929-936.

### 4.2 Apolipoproteins and Apolipoprotein Peptides

The nature of the apolipoproteins expressed recombinantly in a lactic acid bacterium is not critical for success. Virtually any apolipoprotein and/or derivative or analog thereof that provides therapeutic and/or prophylactic benefit as described herein can be expressed in one of more of the members comprising the lactic acid bacteria family. Moreover, any alpha-helical peptide or peptide analog, or any other type of molecule that “mimics” the activity of an apolipoprotein (such as, for example ApoA-I) in that it can activate LCAT or form discoidal particles when associated with lipids, can be expressed recombinantly in a lactic acid bacterium, and is therefore included within the definition of “apolipoprotein.” Examples of suitable apolipoproteins include, but are not limited to, preproapolipoprotein forms of ApoA-I, ApoA-II, ApoA-IV, ApoA-V, and ApoE; pro- and mature forms of human ApoA-I, ApoA-II, ApoA-IV, and ApoE; and active polymorphic forms, isoforms, variants and mutants as well as truncated forms, the most common of which are ApoA-I_{M} (ApoA-I_{M}) and ApoA-I_{P} (ApoA-I_{P}). ApoA-I_{M} is the R173C molecular variant of ApoA-I (see, e.g., Parolini et al., 2003, J Biol Chem. 278(7):4740-6; Calabresi et al., 1999, Biochemistry 38:16307-14; and Calabresi et al., 1997, Biochemistry 36:12428-33). ApoA-I_{P} is the R151 molecular variant of ApoA-I (see, e.g., Daum et al., 1999, J Mol Med. 77(8):614-22). Apolipoproteins mutants containing cysteine residues are also known, and can also be used (see, e.g., U.S. Publication 2003/0181372). The apolipoproteins may be in the form of monomers or dimers, which may be homodimers or heterodimers. For example, homo- and heterodimers (where feasible) of pro- and mature ApoA-I (Duverger et al., 1996, Arterioscler Thromb Vasc Biol. 16(12):1424-29), ApoA-I_{M} (Franceschini et al., 1985, J Biol Chem. 260:1632-35), ApoA-I_{P} (Daum et al., 1999, J Mol Med 77:614-22), ApoA-II (Shelness et al., 1985, J Biol Chem. 260(14):8637-46; Shelness et al., 1984, J Biol Chem. 259(15):9929-35), ApoA-IV (Duverger et al., 1991, Euro J Biochem. 201(2):373-83), ApoE (McLean et al., 1983, J Biol Chem. 258(14):8993-9000), ApoJ and ApoH may be used. The apolipoproteins may include residues corresponding to elements that facilitate their isolation, such as His tags, or other elements designed for other purposes, so long as the apolipoprotein retains some biological activity when included in a complex.

In some embodiments, the nucleotide sequences encoding the apolipoproteins are obtained from humans. Non-limiting examples of human apolipoprotein sequences are disclosed in U.S. Pat. Nos. 5,876,968, 5,643,757, and 5,990,081, and WO 96/37608; the disclosures of which are incorporated herein by reference in their entireties.

In addition to the references above, sequences for human apolipoproteins include sequences available in various sequence databases, such as Genbank. For instance, Genbank Accession Nos. for human ApoA-I include, but are not limited to NP_{—}000030 and AAB59514, P02647, CAA30377, and AAA51746. GenBank Accession No. for human ApoA-II include, but are not limited to NP_{—}001634 and P02652. GenBank Accession Nos. for human ApoA-IV include, but are not limited to, AAB50137, P06727, NP_{—}000473, and NP_{—}001634. GenBank Accession Nos. for human ApoA-V include, but are not limited to, NP_{—}443200, AAB59546, and Q6Q788. GenBank Accession Nos. for human ApoE include, but are not limited to, Q6Q788, P02649, AAB50137, BAA96080, AAG27089, AAL82810, AAB59546, AAB59397, AAH03557, AAD02505, NP_{—}000032, and AAB59518.

In some embodiments, the nucleotide sequences encoding the apolipoproteins are obtained from non-humans (see, e.g., U.S. Publication 2004/0077541, the disclosure of which is incorporated herein by reference in its entirety). Apolipoprotein A-I protein has been identified in a number of non-human animals, for example, cows, horses, sheep, monkeys, baboons, goats, rabbits, dogs, hedgehogs, badgers, mice, rats, cats, guinea pigs, hamsters, duck, chicken, salmon and eel (Brouillette et al., 2001, Biochim Biophys Acta. 1531:4-46; Yu et al., 1991, Cell Struct Funct. 16(4):347-55; Chen and Albers, 1983, Biochim Biophys Acta. 753(1):40-6; Luo et al., 1989, J Lipid Res. 30(11):1735-46; Blaton et al., 1977, Biochemistry 16:2157-63; Sparrow et al., 1995, J Lipid Res. 36(3):485-95; Beaubatie et al., 1986, J Lipid Res. 27:140-49; Januzzi et al., 1992, Genomics 14(4):1081-8; Goulinet and Chapman, 1993, J Lipid Res. 34(6):943-59; Collet et al., 1997, J Lipid Res. 38(4):634-44; and Frank and Marcel, 2000, J Lipid Res. 41(6):853-72).

Apolipoprotein A-I protein derived from non-human animal species are of similar size (Mr≈27,000-28,000) and share considerable homology (Smith et al., 1978, Ann Rev Biochem. 47:751-7). For example, bovine ApoA-I protein comprises 241 amino acid residues and can form a series of repeating amphipathic alpha-helical regions. There are 10 amphipathic alpha-helical regions in bovine ApoA-I protein, typically occurring between residues 43-64, 65-86, 87-97, 98-119, 120-141, 142-163, 164-184, 185-206, 207-217 and 218-241 (see, Sparrow et al., 1992, Biochim BiophysActa. 1123:145-150, and Swaney, 1980, Biochim Biophys Acta. 617:489-502.). An amino acid sequence comparison between human ApoA-I protein (GenBank Accession Nos. XM_{—}52106 or NM_{—}000039) and bovine ApoA-I protein (GenBank Accession No. A56858) using the program BLAST reveals that the sequences are 77% identical (Altschul et al., 1990, J Mol Biol. 215(3):403-10).

Pig (porcine) ApoA-I protein comprises about 264 amino acid residues with a molecular weight of about 30,280. GenBank Accession No. S31394, provides a 264 residue porcine ApoA-I sequence with a molecular weight 30,254, while GenBank Accession No. JT0672 provides a 265 residue porcine ApoA-I protein with a molecular weight of 30,320 (see also, Weiler-Guttler et al., 1990, J Neurochem. 54(2):444-450; Trieu et al., 1993, Gene 123(2):173-79; Trieu et al., 1993, Gene 134(2):267-70).

Chicken ApoA-I precursor has 264 amino acid residues; the sequence is provided at GenBank Accession No. LPCHA1. Jackson et al., have described hen ApoA-I as comprising 234 amino acid residues, having a molecular weight of about 28,000 and differing from human ApoA-I by the presence of isoleucine (Jackson et al., 1976, Biochim Biophys Acta. 420(2):342-9). Yang et al., described mature chicken ApoA-I protein as comprised of 240 amino acid residues with a less than 50% homology with humans (see also, Yang et al., 1987, FEBSLett. 224(2):261-6, Shackelford and Lebherz, 1983, J Biol Chem. 258(11):7175-7180, Banjerjee et al., 1985, J Cell Biol. 101(4):1219-1226, Rajavashisth et al., 1987, J Biol Chem. 262(15):7058-65, Ferrari et al., 1987, Gene 60(1):39-46, Bhattacharyya et al., 1991, Gene 104(2):163-168; Lamon-Fava et al., 1992, J Lipid Res. 33(6):831-42). Circular dichroism studies of chicken ApoA-I protein demonstrate that the protein organizes as a bundle of amphipathic alpha-helices in a lipid free state (Kiss et al., 1999, Biochemistry 38(14):4327-34). A comparison of secondary structural features among chicken, human, rabbit, dog and rat indicates good conservation of ApoA-I secondary structure with human ApoA-I, especially in the N-terminal two-thirds of the protein (Yang et al., 1987, FEBS Lett. 224(2):261-6).

Lipoprotein studies in turkeys have identified an ApoA class of lipoprotein designated in analogy to human ApoA-I and ApoA-II. ApoA-I in turkeys was the major ApoA polypeptide with a molecular weight of about 27,000 (Kelley and Alaupovic, 1976, Atherosclerosis 24(1-2):155-75, Kelley and Alaupovic, 1976, Atherosclerosis 24(1-2):177-87). Duck ApoA-I can comprise about 246 amino acid residues and has a molecular weight of about 28,744 (GenBank Accession No. A61448, Gu et al., 1993, J Protein Chem. 12(5):585-91).

Non-limiting examples of peptides and peptide analogs that correspond to apolipoproteins, as well as agonists that mimic the activity of ApoA-I, ApoA-IM, ApoA-II, ApoA-IV, and ApoE, that are suitable for expression in lactic acid bacteria are disclosed in U.S. Pat. Nos. 6,004,925, 6,037,323, 6,046,166, and 5,840,688, U.S. publications 2004/0266671, 2004/0254120, 2003/0171277, 2003/0045460, and 2003/0087819, the disclosures of which are incorporated herein by reference in their entireties.

### 4.3 Lactic Acid Bacteria Expression Vectors and Regulatory Sequences

The recombinant lactic acid bacterium can comprise at least one constitutive promoter, or at least one regulatable promoter operably linked to the coding nucleotide sequence. As used herein, the term “operably linked” refers to a linkage of nucleotide sequence elements in a functional relationship. For example, a promoter that is “operably linked” means that the regulatory element is in the appropriate location and orientation in relation to a nucleotide coding sequence to control RNA polymerase initiation and expression of the nucleic acid (e.g., it affects the transcription of the coding sequence). The promoter region can be based on a promoter present in any prokaryotic cell, and which promoter is capable of functioning in lactic acid bacteria (e.g., promoting expression of an operably linked nucleic acid sequence), but in some embodiments it is derived from a lactic acid bacterial species. For example, in some embodiments, the promoter region can be derived from a promoter region of Lactococcus lactis including Lactococcus lactis subspecies lactis, e.g. the strain designated MG1363 (also referred to in the literature as Lactococcus lactis subspecies cremoris) (Nauta et al., 1997, Nat Biotechnol. 15:980-983), and Lactococcus lactis subspecies lactis biovar. diacetylactis. Examples of other promoter regions suitable for use in the construction of a recombinant lactic acid bacterium are disclosed in WO 94/16086, including a region comprising the promoter P170, and derivatives thereof, examples of which are disclosed in WO 98/10079 and U.S. application publication No. 2002/0137140, the disclosures of which are incorporated herein by reference in their entirety.

In some embodiments, the lactic acid bacterium used to express a recombinant apolipoprotein can be a variant in which the extracellular housekeeping protease, HtrA has been inactivated. See, e.g., Miyoshi, et al., 2002, Appl Environ Microbiol. 68:3141-3146, the disclosure of which is incorporated herein by reference in its entirety.

In some embodiments, the promoter used in the recombinant lactic acid bacterium can be a regulatable or inducible promoter. The factor(s) regulating or inducing the promoter include any physical and chemical factor that can regulate the activity of a promoter sequence, including, but not limited to, physical conditions, such as temperature and light; chemical substances, such as IPTG, tryptophan, lactate or nisin; and environmental or growth condition factors, such as pH, incubation temperature, and oxygen content. Other conditions for regulating promoter activity can include, among others, a temperature shift eliciting the expression of heat shock genes; the composition of the growth medium such as the ionic strength/NaCl content; accumulation of metabolites, including lactic acid/lactate, intracellularly or in the medium; the presence/absence of essential cell constituents or precursors therefore; and the growth phase or growth rate of the bacterium. See, e.g., U.S. Publication 2002/0137140, the disclosure of which is incorporated herein by reference in its entirety.

A number of inducible gene expression systems for use in lactic acid bacteria have been developed, see, e.g., Kok, 1996, Antonie Van Leeuwenhoek. 70:129-145; Kuipers et al., 1997, Trends Biotechnol. 15:135-40; Djordjevic and Klaenhammer, 1998, Mol Biotechnol. 9:127-139; Kleerebezem, et al., 1997, Appl Environ Microbiol. 63:4581-4584. Useful lactic acid bacterial expression systems include the NICE system (de Ruyter et al., 1996, Appl Environ Microbiol. 62:3662-3667), which is based on genetic elements from a two-component system that controls the biosynthesis of the anti-microbial peptide nisin in L. lactis. Other useful inducible expression systems include the use of genetic elements from the L. lactisbacteriophages φ31 (O'Sullivan et al., 1996, Biotechnology (N.Y.), 14:82-87; and Walker and Klaenhammer, 1998, J Bacteriol. 180:921-931) and rlt (Nauta et al., 1997, Nat Biotechnol. 15:980-983), promoters induced by changes in the environment such as pH (Israelsen et al., 1995, Appl Environ Microbiol. 61:2540-2547), Zn²⁺ (Llull and Poquet, 2004, Appl Environ Microbiol. 70:5398-5406), salt concentration (Sanders et al., 1998, Mol Gen Genet, 257:681-685), and metabolites produced by the host cell or by conditions naturally occurring during host cell growth. Such promoter include, among others, the pH inducible P170 promoter and derivatives thereof, as disclosed in WO 94/16086, WO 98/10079, U.S. application publication No. 2002/0137140, and Madsen et al., 1999, Mol Microbiol. 107:75-87.

In some embodiments, the promoter and the nucleotide sequence coding for the apolipoprotein can be introduced into the lactic acid bacterium on an autonomously replicating replicon, such as a plasmid, a transposable element, a bacteriophage, or a cosmid. In some embodiments, the promoter and the apolipoprotein nucleotide coding sequence can be introduced under conditions in which the apolipoprotein nucleotide coding sequence becomes integrated into the lactic acid bacterium cell chromosome, so as to provide stable maintenance in the bacterium of the apolipoprotein nucleotide coding sequence. Integration can be affected by integration systems based on, among others, homologous recombination, transposons, conjugal transfers, and phage integrases (see, e.g., Frazier et al., 2003, Appl Environ Microbiol. 69(2):1121-8.; Christiansen et al., 1994, J Bacteriol. 176(4):1069-76; Romero et al., 1992, Appl Environ Microbiol. 58(2):699-702.; Romero et al., 1991, J Bacteriol. 173(23):7599-606.; Leenhouts et al., 1991, Appl Environ Microbiol. 57(9):2562-7.; Leenhouts et al., 1990, Appl Environ Microbiol. 56(9):2726-2735; Chopin et al., 1989, Appl Environ Microbiol. 55(7):1769-74; and Scheirlinck et al., 1989, Appl Environ Microbiol 55(9):2130-7).

In other embodiments, the apolipoprotein nucleotide coding sequence can be introduced into the lactic acid bacterium cell chromosome at a location where it becomes operably linked to a promoter naturally occurring in the chromosome of the selected host organism (see, e.g., Rauch et al., 1992, J Bacteriol. 174(4):1280-7; Israelsen et al., 1993, Appl Environ Microbiol. 59(1):21-26.; and Maguin et al., 1996, J Bacteriol. 178(3):931-5).

In some embodiments, the apolipoprotein nucleotide coding sequence is operably linked to a nucleotide sequence coding for a signal peptide (SP) enabling the gene product to be secreted out of the bacterium and into the culture medium. Signal peptides suitable for use in lactic acid bacteria, including Usp 45 are disclosed in U.S. Publication 2002/0137140, the disclosure of which is incorporated herein by reference in its entirety.

In some embodiments, additional nucleotide sequences, such as those used to improve the production and secretion of heterologous proteins in lactic acid bacteria can be used in the methods and compositions described herein. For example, in some embodiments, nucleotide sequences coding for staphyloccal nuclease (Nuc) and the synthetic propeptide LEISSTCDA, can be linked to a nucleotide sequence coding for an apolipoprotein. See, e.g., Nouaille et al., 2005, Braz J Med Biol Res. 38:353-359, the disclosure of which is incorporated herein by reference in its entirety.

In some embodiments, expression vectors developed for use in lactic acid bacteria can be used to express recombinant apolipoprotein in a lactic acid bacterium, including, but not limited to, pLF22 (see, e.g., Trakanov, et al., 2004, Microbiology 73:170-175) and pTREX (see, e.g., Reuter, et al., 2003, “Vaccine Protocols,” In Methods in Molecular Medicine 87:101-114).

In various embodiments, the lactic acid bacteria comprising a nucleotide sequence encoding an apolipoprotein can be cultivated, for example as disclosed in U.S. Publication 2002/0137140, to produce endotoxin-free apolipoprotein. The methods can comprise culturing the transformed lactic acid bacteria under conditions suitable for the expression of the apolipoprotein, and recovering the apolipoprotein from the transformed lactic acid bacteria. The recombinant cells and/or the apolipoprotein can be harvested using conventional techniques known to those skilled in the art. See, e.g., U.S. application publication No. 2002/0137140, the disclosure of which is incorporated herein by reference in its entirety.

In some embodiments, short chain acyl phospholipids are added to the medium (i.e. fermentation medium) used to cultivate the lactic acid bacteria comprising the recombinant apolipoprotein (as described above). The lactic acid bacteria can utilize the short chain acyl phospholipids as a nutrient source. Additionally, the short chain acyl phospholipids can be used as an aid to solubilize the expressed apolipoprotein. The short chain acyl phospholipids can be easily removed by adding a phospholipase that clips the acyl chain, liberating a short chain fatty acid and a short chain lysoPL. As the short chain fatty acid and lysoPL are soluble, the apolipoprotein can be precipitated and purified.

### 4.4 Recombinant Apolipoprotein-Lipid Complexes

In some embodiments, the recombinant apolipoproteins described herein can be formulated and administered in an apolipoprotein-lipid complex. This approach has several advantages since the complex should have an increased half-life in the circulation, particularly when the complex has a similar size and density to HDL, and especially the pre-beta-1 or pre-beta-2 HDL populations. The apolipoprotein-lipid complexes can conveniently be prepared by any of a number of methods described below. See also U.S. Pat. No. 6,004,925, the disclosure of which is incorporated herein by reference in its entirety. Stable preparations having a long shelf life may be made by lyophilization. The lyophilized apolipoprotein-lipid complexes can be used to prepare bulk for pharmaceutical reformulation, or to prepare individual aliquots or dosage units which can be reconstituted by rehydration with sterile water or an appropriate buffered solution prior to administration to a subject.

A variety of methods well known to those skilled in the art can be used to prepare the apolipoprotein-lipid vesicles or complexes. To this end, a number of available techniques for preparing liposomes or proteoliposomes can be used. For example, the apolipoprotein can be cosonicated (using a bath or probe sonicator) with appropriate lipids to form complexes. Alternatively the apolipoprotein can be combined with preformed lipid vesicles resulting in the spontaneous formation of apolipoprotein-lipid complexes. In yet other embodiments, the apolipoprotein-lipid complexes can be formed by a detergent dialysis method; e.g., a mixture of the apolipoprotein, lipid and detergent is dialyzed to remove the detergent and reconstitute or form apolipoprotein-lipid complexes (see, e.g., Jonas et al., 1986, Methods in Enzymol. 128:553-582).

While the foregoing approaches are feasible, each method presents its own peculiar production problems in terms of cost, yield, reproducibility and safety. A simple method for preparing apolipoprotein-phospholipid complexes which have characteristics similar to HDL is described in U.S. Pat. No. 6,004,925, the disclosure of which is incorporated herein by reference in its entirety.

The lyophilized product can be reconstituted in order to obtain a solution or suspension of the peptide-lipid complex. To this end, the lyophilized powder is rehydrated with an aqueous solution to a suitable volume (often 5 mgs peptide/ml which is convenient for intravenous injection). In some embodiments, the lyophilized powder is rehydrated with phosphate buffered saline or a physiological saline solution. The mixture can be agitated or vortexed to facilitate rehydration, and in most cases, the reconstitution step can be conducted at a temperature equal to or greater than the phase transition temperature of the lipid component of the complexes.

An aliquot of the resulting reconstituted preparation can be characterized to confirm that the complexes in the preparation have the desired size distribution; e.g., the size distribution of HDL. An exemplary method for this purpose is gel filtration chromatography. In the working examples described infra, a Pharmacia Superose 6 FPLC gel filtration chromatography system was used. The buffer used contains 150 mM NaCl in 50 mM phosphate buffer, pH 7.4. A typical sample volume is 20 to 200 microliters of complexes containing 5 mgs peptide/ml. The column flow rate is 0.5 mls/min. A series of proteins of known molecular weight and Stokes' diameter, as well as human HDL, can be used as standards to calibrate the column. The proteins and lipoprotein complexes can be monitored by absorbance or scattering of light of wavelength 254 or 280 nm

The recombinant apolipoproteins can be complexed with a variety of lipids, including saturated, unsaturated, natural and synthetic lipids and/or phospholipids. Suitable lipids include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, sphingomyelin, sphingolipids, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, dipalmitoylsphingomyelin, distearoylsphingomyelin, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio)hexyl ether glycolipids, and cholesterol and its derivatives.

In other embodiments, recombinant apolipoprotein-lipid complexes can be made by complexing the recombinant apolipoproteins with the lipids disclosed in U.S. application Ser. No. 60/665,180, entitled “Charged Lipoprotein Complexes and Their Uses,” filed Mar. 24, 2005, and International application No. PCT/IB2006/000635.

### 4.5 Pharmaceutical Compositions

The pharmaceutical compositions contemplated by the disclosure comprise a recombinant apoliprotein as described herein, or a recombinant apolipoprotein-lipid complex as the active ingredient in a pharmaceutically acceptable carrier suitable for administration and delivery in vivo. In embodiments using peptide mimetic apolipoproteins, the peptide mimetic apolipoproteins can be included in the compositions in either the form of free acids or bases, or in the form of pharmaceutically acceptable salts. Modified proteins such as amidated, acylated, acetylated or pegylated proteins, can also be used.

Injectable compositions include sterile suspensions, solutions or emulsions of the active ingredient in aqueous or oily vehicles. The compositions can also comprise formulating agents, such as suspending, stabilizing and/or dispersing agent. The compositions for injection can be presented in unit dosage form, e.g., in ampules or in multidose containers, and can comprise added preservatives. For infusion, a composition can be supplied in an infusion bag made of material compatible with charged lipoprotein complexes, such as ethylene vinyl acetate or any other compatible material known in the art.

Alternatively, the injectable compositions can be provided in powder form for reconstitution with a suitable vehicle, including but not limited to, sterile pyrogen free water, buffer, dextrose solution, etc., before use. To this end, the recombinant apolipoprotein can be lyophilized, or co-lyophilized apolipoprotein-lipid complexes can be prepared. The stored compositions can be supplied in unit dosage forms and reconstituted prior to use in vivo.

For prolonged delivery, the active ingredient can be formulated as a depot composition, for administration by implantation; e.g., subcutaneous, intradermal, or intramuscular injection. Thus, for example, recombinant apolipoprotein-lipid complex or recombinant apolipoprotein alone can be formulated with suitable polymeric or hydrophobic materials (e.g., as an emulsion in an acceptable oil) or in phospholipid foam or ion exchange resins.

Alternatively, transdermal delivery systems manufactured as an adhesive disc or patch that slowly releases the active ingredient for percutaneous absorption can be used. To this end, permeation enhancers can be used to facilitate transdermal penetration of the active ingredient. A particular benefit can be achieved by incorporating the charged complexes described herein into a nitroglycerin patch for use in patients with ischemic heart disease and hypercholesterolemia.

Alternatively, the delivery could be done locally or intramurally (within the vessel wall) using a catheter or perfusor (see, e.g., U.S. application publication No. 2003/0109442).

The compositions can, if desired, be presented in a pack or dispenser device that may comprise one or more unit dosage forms comprising the active ingredient. The pack can for example comprise metal or plastic foil, such as a blister pack. The pack or dispenser device can be accompanied by instructions for administration.

### 4.6 Methods of Treatment

The recombinant apolipoproteins and/or recombinant apolipoprotein-lipid complexes and compositions described herein can be used for virtually every purpose lipoprotein complexes have been shown to be useful. In some embodiments, the complexes and compositions can be used to treat or prevent dyslipidemia and/or virtually any disease, condition and/or disorder associated with dyslipidemia. As used herein, the terms “dyslipidemia” or “dyslipidemic” refer to an abnormally elevated or decreased level of lipid in the blood plasma, including, but not limited to, the altered level of lipid associated with the following conditions: coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

Diseases associated with dyslipidemia include, but are not limited to coronary heart disease, coronary artery disease, acute coronary syndrome, cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

In some embodiments, the methods encompass a method of treating or preventing a disease associated with dyslipidemia, comprising administering to a subject a recombinant apolipoprotein and/or recombinant apolipoprotein-lipid complex in an amount effective to achieve a serum level of free or complexed apolipoprotein for at least one day following administration that is in the range of about 10 mg/dL to 300 mg/dL higher than a baseline (initial) level prior to administration.

In other embodiments, the methods encompass a method of treating or preventing a disease associated with dyslipidemia, comprising administering to a subject a recombinant apolipoprotein and/or recombinant apolipoprotein-lipid complex in an amount effective to achieve a circulating plasma concentrations of a HDL-cholesterol fraction for at least one day following administration that is at least about 10% higher than an initial HDL-cholesterol fraction prior to administration.

In other embodiments, the methods encompass a method of treating or preventing a disease associated with dyslipidemia, comprising administering to a subject a charged lipoprotein complex or composition described herein in an amount effective to achieve a circulating plasma concentration of a HDL-cholesterol fraction that is between 30 and 300 mg/dL between 5 minutes and 1 day after administration.

In other embodiments, the methods encompass a method of treating or preventing a disease associated with dyslipidemia, comprising administering to a subject a recombinant apolipoprotein and/or recombinant apolipoprotein-lipid complex in an amount effective to achieve a circulating plasma concentration of cholesteryl esters that is between 30 and 300 mg/dL between 5 minutes and 1 day after administration.

In other embodiments, the methods encompasses a method at treating or protecting a disease associated with dyslipidemia, comprising administering to a subject a recombinant apolipoprotein and/or recombinant apolipoprotein-lipid complex in an amount effective to achieve an increase in fecal cholesterol excretion for at least one day following administration that is at least about 10% above a baseline (initial) level prior to administration.

The recombinant apolipoprotein and/or recombinant apolipoprotein-lipid complexes or compositions described herein can be used alone or in combination therapy with other drugs used to treat or prevent the foregoing conditions. Such therapies include, but are not limited to simultaneous or sequential administration of the drugs involved. For example, in the treatment of hypercholesterolemia or atherosclerosis, recombinant apolipoproteins and/or recombinant apolipoprotein-lipid complexes can be administered with any one or more of the cholesterol lowering therapies currently in use; e.g., bile-acid resins, niacin, statins, inhibitors of cholesterol absorption and/or fibrates. Such a combined regimen can produce particularly beneficial therapeutic effects since each drug acts on a different target in cholesterol synthesis and transport; i.e., bile-acid resins affect cholesterol recycling, the chylomicron and LDL population; niacin primarily affects the VLDL and LDL population; the statins inhibit cholesterol synthesis, decreasing the LDL population (and perhaps increasing LDL receptor expression); whereas the charged lipoprotein complexes described herein affect RCT, increase HDL, and promote cholesterol efflux.

In other embodiments, the recombinant apolipoproteins and/or recombinant apolipoprotein-lipid complexes can be used in conjunction with fibrates to treat or prevent coronary heart disease; coronary artery disease; cardiovascular disease, hypertension, restenosis, vascular or perivascular diseases; dyslipidemic disorders; dyslipoproteinemia; high levels of low density lipoprotein cholesterol; high levels of very low density lipoprotein cholesterol; low levels of high density lipoproteins; high levels of lipoprotein Lp(a) cholesterol; high levels of apolipoprotein B; atherosclerosis (including treatment and prevention of atherosclerosis); hyperlipidemia; hypercholesterolemia; familial hypercholesterolemia (FH); familial combined hyperlipidemia (FCH); lipoprotein lipase deficiencies, such as hypertriglyceridemia, hypoalphalipoproteinemia, and hypercholesterolemialipoprotein.

The recombinant apolipoproteins and/or recombinant apolipoprotein-lipid complexes can be administered by any suitable route that ensures bioavailability in the circulation. For example, the recombinant apolipoproteins and/or recombinant apolipoprotein-lipid complexes can be administered in dosages that increase the small HDL fraction, for example, the pre-beta, pre-gamma and pre-beta-like HDL fraction, the alpha HDL fraction, the HDL3 and/or the HDL2 fraction. In some embodiments, the dosages are effective to achieve atherosclerotic plaque reduction as measured by, for example, imaging techniques such as magnetic resonance imaging (MRI) or intravascular ultrasound (IVUS). Parameters to follow by IVUS include, but are not limited to, change in percent atheroma volume from baseline and change in total atheroma volume. Parameters to follow by MRI include, but are not limited to, those for IVUS and lipid composition and calcification of the plaque.

The plaque regression can be measured using the patient as its own control, time zero versus time t at the end of the last infusion, or within weeks after the last infusion, or within 3 months, 6 months, or 1 year after the start of therapy.

Administration can best be achieved by parenteral routes of administration, including intravenous (IV), intramuscular (IM), intradermal, subcutaneous (SC), and intraperitoneal (IP) injections. In certain embodiments, administration is by a perfusor, an infiltrator or a catheter. In some embodiments, the charged lipoprotein complexes are administered by injection, by a subcutaneously implantable pump or by a depot preparation, in amounts that achieve a circulating serum concentration equal to that obtained through parenteral administration. The complexes could also be absorbed in, for example, a stent or other device.

Administration can be achieved through a variety of different treatment regimens. For example, several intravenous injections can be administered periodically during a single day, with the cumulative total volume of the injections not reaching the daily toxic dose. Alternatively, one intravenous injection can be administered about every 3 to 15 days, preferably about every 5 to 10 days, and most preferably about every 10 days. In yet another alternative, an escalating dose can be administered, starting with about 1 to 5 doses at a dose between (50-200 mg) per administration, then followed by repeated doses of between 200 mg and 1 g per administration. Depending on the needs of the patient, administration can be by slow infusion with a duration of more than one hour, by rapid infusion of one hour or less, or by a single bolus injection.

In some embodiments, administration can be done as a series of injections and then stopped for 6 months to 1 year, and then another series started. Maintenance series of injections can then be administered every year or every 3 to 5 years. The series of injections could be done over a day (perfusion to maintain a specified plasma level of complexes), several days (e.g., four injections over a period of eight days) or several weeks (e.g., four injections over a period of four weeks), and then restarted after six months to a year.

Other routes of administration can be used. For example, absorption through the gastrointestinal tract can be accomplished by oral routes of administration (including but not limited to ingestion, buccal and sublingual routes) provided appropriate formulations (e.g., enteric coatings) are used to avoid or minimize degradation of the active ingredient, e.g., in the harsh environments of the oral mucosa, stomach and/or small intestine. Alternatively, administration via mucosal tissue such as vaginal and rectal modes of administration may be utilized to avoid or minimize degradation in the gastrointestinal tract. In other embodiments, the formulations of the invention can be administered transcutaneously (e.g., transdermally), or by inhalation. It will be appreciated that the preferred route may vary with the condition, age and compliance of the recipient.

The actual dose of a recombinant apolipoprotein and/or recombinant apolipoprotein-lipid complex or composition can vary with the route of administration.

Toxicity and therapeutic efficacy of the various recombinant apolipoproteins and/or recombinant apolipoprotein-lipid complexes can be determined using standard pharmaceutical procedures in cell culture or experimental animals for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Recombinant apolipoproteins and/or recombinant apolipoprotein-lipid complexes that exhibit large therapeutic indices are preferred. Non-limiting examples of parameters that can be followed include liver function transaminases (no more than 2×normal baseline levels). This is an indication that too much cholesterol is brought to the liver and that the liver cannot assimilate such an amount. The effect on red blood cells could also be monitored, as mobilization of cholesterol from red blood cells causes them to become fragile, or affect their shape.

Patients can be treated from a few days to several weeks before a medical act (e.g., preventive treatment), or during or after a medical act. Administration can be concomitant to or contemporaneous with another invasive therapy, such as, angioplasty, carotid ablation, rotoblader or organ transplant (e.g., heart, kidney, liver, etc.).

In certain embodiments, recombinant apolipoproteins and/or recombinant apolipoprotein-lipid complexes are administered to a patient whose cholesterol synthesis is controlled by a statin or a cholesterol synthesis inhibitor. In other embodiments, recombinant apolipoproteins and/or recombinant apolipoprotein-lipid complexes are administered to a patient undergoing treatment with a binding resin, e.g., a semi-synthetic resin such as cholestyramine, or with a fiber, e.g., plant fiber, to trap bile salts and cholesterol, to increase bile acid excretion and lower blood cholesterol concentrations.

### 4.7 Other Uses

The recombinant apolipoproteins and/or recombinant apolipoprotein-lipid complexes and compositions described herein can be used in assays in vitro to measure serum HDL, e.g, for diagnostic purposes. Because ApoA-I, ApoA-II and Apo peptides associate with the HDL component of serum, recombinant apolipoproteins and/or recombinant apolipoprotein-lipid complexes can be used as “markers” for the HDL population, and the pre-betal and pre-beta2 HDL populations. Moreover, the recombinant apolipoproteins and/or recombinant apolipoprotein-lipid complexes can be used as markers for the subpopulation of HDL that are effective in RCT. To this end, recombinant apolipoproteins and/or recombinant apolipoprotein-lipid complexes can be added to or mixed with a patient serum sample; after an appropriate incubation time, the HDL component can be assayed by detecting the incorporated recombinant apolipoproteins and/or recombinant apolipoprotein-lipid complexes. This can be accomplished using labeled recombinant apolipoproteins and/or recombinant apolipoprotein-lipid complexes (e.g., radiolabels, fluorescent labels, enzyme labels, dyes, etc.), or by immunoassays using antibodies (or antibody fragments) specific for recombinant apolipoproteins and/or recombinant apolipoprotein-lipid complexes.

Alternatively, labeled recombinant apolipoproteins and/or recombinant apolipoprotein-lipid complexes can be used in imaging procedures (e.g., CAT scans, MRI scans) to visualize the circulatory system, or to monitor RCT, or to visualize accumulation of HDL at fatty streaks, atherosclerotic lesions, and the like, where the HDL should be active in cholesterol efflux

All cited references are incorporated herein by reference in their entirety and for all purposes to the same extent as if each individual publication, patent or patent application was specifically and individually indicated to be incorporated herein by reference in its entirety for all purposes.

The citation of any publication is for its disclosure prior to the filing date and should not be construed as an admission that the present invention is not entitled to antedate such publication by virtue of prior invention.

Many modifications and variations of this invention can be made without departing from its spirit and scope, as will be apparent to those skilled in the art. The specific embodiments described are offered by way of example only, and the invention is to be limited only by the terms of the appended claims along with the full scope of equivalents to which such claims are entitled.

What is claimed is:

1. An expression vector capable of expression in a lactic acid bacterium, comprising a nucleotide coding sequence encoding an apolipoprotein, and one or more regulatory nucleotide sequences operably linked thereto to control the expression of the nucleotide coding sequence.

2. The expression vector according to claim 1 in which the nucleotide coding sequence encodes a human apolipoprotein.

3. The expression vector according to claim 2 in which the nucleotide coding sequence encodes a human apolipoprotein selected from preproapoliprotein, preproApoA I, proApoA I, ApoA I, preproApoA II, proApoA II, ApoA II, preproApoA -IV, proApoA IV, ApoA IV, ApoA V, preproApoE, proApoE, ApoE, preproApoA IMilano, proApoA IMilano, ApoA IMilano, preproApoA IParis, proApoA IParis, and ApoA IParis.

4. The expression vector according to claim 1 in which one of the regulatory nucleotide sequences comprises a constitutive promoter operably linked to the nucleotide coding sequence.

5. The expression vector according to claim 1 in which one of the regulatory nucleotide sequences comprises a regulatable promoter operably linked to the nucleotide coding sequence.

6. The expression vector according to claim 4 or 5 in which the promoter is derived from a lactic acid bacterium.

7. The expression vector according to claim 6 in which the promoter is regulated by a factor selected from the group consisting of pH, temperature, and oxygen.

8. The expression vector according to claim 5 in which the regulatable promoter is the P170 promoter.

9. The expression vector according to any one of claims 1-8 in which the vector is an autonomously replicating replicon.

10. The expression vector according to claim 9 in which the vector is selected from a plasmid, a transposable element, a bacteriophage, or a cosmid.

11. The expression vector according to any one of claims 1-8 in which the vector is stably integrated into the host cell chromosome.

12. A lactic acid bacterium comprising the nucleotide coding sequence according to any one of claims 1, 2 or 3.

13. A lactic acid bacterium comprising the expression vector according to any one of claims 1-10.

14. A lactic acid bacterium expressing a protein encoded by the nucleotide coding sequence according to any one of claims 1-10.

15. The lactic acid bacterium according to any one of claims 12, 13 or 14 selected from Lactococcus spp., Streptococcus spp., Lactobacillus spp., Leuconostoc spp., Pediococcus spp., Brevibacterium spp. and Propionibacterium spp.

16. An endotoxin free apolipoprotein produced by lactic acid bacteria transformed with the expression vector according to any one of claims 1-11.

17. A method of producing an endotoxin-free apolipoprotein, comprising
a) culturing a lactic acid bacteria comprising a nucleotide coding sequence encoding apolipoprotein under conditions suitable for the expression of the apolipoprotein; and
b) recovering the apolipoprotein from the transformed lactic acid bacteria.

18. The method according to claim 17 in which the lactic acid bacteria is transformed with the expression vector of any one of claims 1-11.

19. The method according to claim 17 in which the nucleotide coding sequence encodes a human apolipoprotein.

20. The method according to claim 19 in which the nucleotide coding sequence encodes a human lipoprotein selected from preproapoliprotein, preproApoA I, proApoA I, ApoA I, preproApoA II, proApoA II, ApoA II, preproApoA -IV, proApoA IV, ApoA IV, ApoA V, preproApoE, proApoE, ApoE, preproApoA IMilano, proApoA IMilano, ApoA IMilano, preproApoA IParis, proApoA IParis, and ApoA IParis.

21. The method according to claim 17 in which one of the regulatory nucleotide sequences comprises a constitutive promoter operably linked to the nucleotide coding sequence.

22. The method according to claim 17 in which one of the regulatory nucleotide sequences comprises a regulatable promoter operably linked to the nucleotide coding sequence.

23. The method according to claim 21 or 22 in which the promoter is derived from a lactic acid bacterium.

24. The method according to claim 23 in which the promoter is regulated by a factor selected from the group consisting of pH, temperature, and oxygen.

25. The method according to claim 22 in which the regulatable promoter comprises the P170 promoter.

26. The method according to any one of claims 17-25 in which the vector comprises an autonomously replicating replicon.

27. The method according to claim 26 in which the vector is selected from a plasmid, a transposable element, a bacteriophage or a cosmid.

28. The method according to any one of claims 17-25 in which the vector is stably integrated into the host cell chromosome.

29. The method according to claim 17 in which the lactic acid bacterium is selected from Lactococcus spp., Streptococcus spp., Lactobacillus spp., Leuconostoc spp., Pediococcus spp., Brevibacterium spp. and Propionibacterium spp.
